(19) United States
(12) Patent Application Publication
(10) Pub. No.: US 2026/0330651 A1
(43) Pub. Date: Oct. 22, 2026

(54) BRAIN-COMPUTER INTERFACE-BASED VISUAL STIMULUS PRESENTATION DEVICE AND METHOD

(71) Applicants: Hyundai Motor Company, Seoul (KR); Kia Corporation, Seoul (KR)

(72) Inventors: Hoseung Cha, Hwaseong-si (KR); Seungkyu Nam, Hwaseong-si (KR); Taejun Lee, Hwaseong-si (KR)

(21) Appl. No.: 19/395,566

(22) Filed: Nov. 20, 2025

(30) Foreign Application Priority Data
Apr. 18, 2025 (KR) 10-2025-0050999

(51) Int. Cl. G06T 7/215; G06F 3/01

(52) U.S. Cl. CPC G06T 7/215; G06F 3/015; G06T 2207/20221

(57) ABSTRACT

A BCI-based visual stimulus presentation method, performed by a computing device including a processor and a memory, may include obtaining a motion image from a video, by the processor, generating a first visual stimulus image divided into a target region and a background region through image segmentation based on the motion image, by the processor, generating a second visual stimulus image by synthesizing the motion image and the first visual stimulus image through image blending, by the processor, and simultaneously presenting the first visual stimulus that blinks the target region at a first cycle and the second visual stimulus in which the motion image is sequentially output in a second cycle through the second visual stimulus image, by the processor.

### CROSS-REFERENCE TO RELATED APPLICATION

This application claims, under 35 U.S.C. § 119(a), the benefit of Korean Patent Application No. 10-2025-0050999 filed Apr. 18, 2025, in the Korean Intellectual Property Office, the entire contents of which is incorporated herein by reference.

### BACKGROUND

### (a) Technical Field

The present disclosure relates to a brain-computer interface (BCI)-based visual stimulus presentation device and method and, more particularly, to a BCI-based visual stimulus presentation device and method for active observation rehabilitation.

### (b) Background

Stroke is a neurological symptom that occurs when a part of the brain is damaged due to a blockage (cerebral infarction) or rupture (cerebral hemorrhage) of a blood vessel supplying blood to that part of the brain. Functions of the brain damaged area may be impaired, and rehabilitation treatment may be performed to minimize these impairments.

Action observation (AO) using a mirror neuron system (MNS) has been proposed for rehabilitation treatment. Recently, more effective MNS-based AO rehabilitation technology is being developed by linking BCI technology.

Steady-state visual evoked potential (SSVEP)-BCI technology uses an SSVEP for a BCI. SSVEP is a waveform generated in the visual cortex (occipital lobe) occurring when gazing a visual stimulus that blinks at a constant frequency, and the same frequency components are observed in brain waves.

The visual stimulus evoking steady-state motion visual evoked potential (SSMVEP) has recently been proposed as a method of inducing SSVEP by utilizing stimulus motion.

### SUMMARY

The present disclosure attempts to provide a BCI-based visual stimulus presentation device and method capable of simultaneously using SSMVEP through active observation and SSVEP through visual stimulus that blinks only a target motion part.

The present disclosure attempts to provide a BCI-based visual stimulus presentation device and method capable of inducing SSVEP/SSMVEP only when the patient looks at the motion part required for rehabilitation, and identifying the rehabilitation concentration of the patient by utilizing SSVEP/SSMVEP responsiveness.

A BCI-based visual stimulus presentation method, performed by a computing device including a processor and a memory, may include obtaining a motion image from a video, by the processor, generating a first visual stimulus image divided into a target region and a background region through image segmentation based on the motion image, by the processor, generating a second visual stimulus image by synthesizing the motion image and the first visual stimulus image through image blending, by the processor, and simultaneously presenting the first visual stimulus that blinks the target region at a first cycle and the second visual stimulus in which the motion image is sequentially output in a second cycle through the second visual stimulus image, by the processor.

The obtaining the motion image from the video may include extracting a plurality of motion images over time from a motion video photographed for action observation (AO), by the processor.

The generating the first visual stimulus image divided into the target region and the background region through image segmentation based on the motion image may include storing position information of the target region, by the processor, and changing a color of the target region to a different color from a color of the background region, by the processor.

The generating the second visual stimulus image by synthesizing the motion image and the first visual stimulus image through image blending may include writing shader code for the image blending, by the processor, through Equation 1 of X_{new}=X_{org}+αX_{blend}, where, X_{new} is the second visual stimulus image, X_org is the motion image, X_blend is the first visual stimulus image, and α is a synthesis coefficient.

The generating the second visual stimulus image by synthesizing the motion image and the first visual stimulus image through image blending may further include enabling the second visual stimulus image to output the motion image as is when α is 0, and enabling the second visual stimulus image to brightly display only the target region when α is 1, by the processor.

The simultaneously presenting the first visual stimulus that blinks the target region at the first cycle and the second visual stimulus in which the motion image is sequentially output in the second cycle through the second visual stimulus image may include controlling the synthesis coefficient multiplied to the first visual stimulus image and an output frequency of the motion image, by the processor.

The controlling the synthesis coefficient multiplied to the first visual stimulus image and the output frequency of the motion image may include determining a first frequency of the first visual stimulus through the synthesis coefficient, by the processor, and determining the output frequency of the motion image as a second frequency that is the same as or different from the first frequency, by the processor.

The simultaneously presenting the first visual stimulus that blinks the target region at the first cycle and the second visual stimulus in which the motion image is sequentially output in the second cycle through the second visual stimulus image may include variably determining a first frequency for determining the first cycle and a second frequency for determining the second cycle, depending on a presentation purpose of the second visual stimulus image, by the processor.

The variably determining the first frequency for determining the first cycle and the second frequency for determining the second cycle, depending on the presentation purpose of the second visual stimulus image may include determining the first frequency to be greater than the second frequency, when a presentation purpose of the first visual stimulus is purposed to induce SSVEP and the presentation purpose of the second visual stimulus is purposed to induce concentration, by the processor.

The simultaneously presenting the first visual stimulus that blinks the target region at the first cycle and the second visual stimulus in which the motion image is sequentially output in the second cycle through the second visual stimulus image may include providing a notification to induce concentration, when an SSVEP or SSMVEP response size according to the second visual stimulus is smaller than or equal to a preset criterion, by the processor.

A BCI-based visual stimulus presentation device for active observation rehabilitation by executing program code loaded on one or more memory devices through one or more processors, where the program code may include, when executed, obtaining a motion image from a video, generating a first visual stimulus image divided into a target region and a background region through image segmentation based on the motion image, generating a second visual stimulus image by synthesizing the motion image and the first visual stimulus image through image blending, and simultaneously presenting the first visual stimulus that blinks the target region at a first cycle and the second visual stimulus in which the motion image is sequentially output in a second cycle through the second visual stimulus image.

The obtaining the motion image from the video may include extracting a plurality of motion images over time from a motion video photographed for action observation (AO).

The generating the first visual stimulus image divided into the target region and the background region through image segmentation based on the motion image may include storing position information of the target region, and changing a color of the target region to a different color from a color of the background region.

The generating the second visual stimulus image by synthesizing the motion image and the first visual stimulus image through image blending may include writing shader code for the image blending through Equation 1 of X_{new}=X_{org}+αX_{blend}, where X_{new} is the second visual stimulus image, X_org is the motion image, X_blend is the first visual stimulus image, and α is a synthesis coefficient.

The generating the second visual stimulus image by synthesizing the motion image and the first visual stimulus image through image blending may further include enabling the second visual stimulus image to output the motion image as is when α is 0, and enabling the second visual stimulus image to brightly display only the target region when α is 1.

The simultaneously presenting the first visual stimulus that blinks the target region at the first cycle and the second visual stimulus in which the motion image is sequentially output in the second cycle through the second visual stimulus image may include controlling the synthesis coefficient multiplied to the first visual stimulus image and an output frequency of the motion image.

The controlling the synthesis coefficient multiplied to the first visual stimulus image and the output frequency of the motion image may include determining a first frequency of the first visual stimulus through the synthesis coefficient, and determining the output frequency of the motion image as a second frequency that is the same as or different from the first frequency.

The simultaneously presenting the first visual stimulus that blinks the target region at the first cycle and the second visual stimulus in which the motion image is sequentially output in the second cycle through the second visual stimulus image may include variably determining a first frequency for determining the first cycle and a second frequency for determining the second cycle, depending on a presentation purpose of the second visual stimulus image.

The variably determining the first frequency for determining the first cycle and the second frequency for determining the second cycle, depending on the presentation purpose of the second visual stimulus image may include determining the first frequency to be greater than the second frequency, when a presentation purpose of the first visual stimulus is purposed to induce SSVEP and the presentation purpose of the second visual stimulus is purposed to induce concentration.

The simultaneously presenting the first visual stimulus that blinks the target region at the first cycle and the second visual stimulus in which the motion image is sequentially output in the second cycle through the second visual stimulus image may further include providing a notification to induce concentration, when an SSVEP or SSMVEP response size according to the second visual stimulus is smaller than or equal to a preset criterion.

A BCI-based visual stimulus presentation device and method according to an embodiment can simultaneously use SSMVEP through active observation and SSVEP through visual stimulus that blinks only a target motion part, to induce SSVEP/SSMVEP only when the patient looks at the motion part required for rehabilitation, thereby identifying the rehabilitation concentration of the patient by utilizing SSVEP/SSMVEP responsiveness.

According to an aspect of the present disclosure, a brain-computer interface (BCI)-based visual stimulus presentation method is provided. The method may comprise, using a computing device comprising a processor and a memory, obtaining a motion image from a video, generating a first visual stimulus image divided into a target region and a background region, generating a second visual stimulus image by synthesizing the motion image and the first visual stimulus image, and simultaneously presenting the first visual stimulus that blinks the target region at a first cycle and the second visual stimulus in which the motion image is sequentially output in a second cycle through the second visual stimulus image.

According to an exemplary embodiment, the obtaining the motion image from the video may comprise extracting a plurality of motion images over time from a motion video photographed for action observation (AO).

According to an exemplary embodiment, the first visual stimulus image may be generated through image segmentation based on the motion image, and the second visual stimulus image may be generated through image blending.

According to an exemplary embodiment, the generating the first visual stimulus image divided into the target region and the background region through image segmentation based on the motion image may comprise storing position information of the target region, and changing a color of the target region to a different color from a color of the background region.

According to an exemplary embodiment, the generating the second visual stimulus image by synthesizing the motion image and the first visual stimulus image through image blending may comprise writing shader code for the image blending, by the processor, using X_new=X_org+αX_blend. X_new is the second visual stimulus image, X_org is the motion image, X_blend is the first visual stimulus image, and α is a synthesis coefficient.

According to an exemplary embodiment, the generating the second visual stimulus image by synthesizing the motion image and the first visual stimulus image through image blending further may comprise enabling the second visual stimulus image to output the motion image as is when α is 0, and enabling the second visual stimulus image to brightly display only the target region when α is 1.

According to an exemplary embodiment, the simultaneously presenting the first visual stimulus that blinks the target region at the first cycle and the second visual stimulus in which the motion image is sequentially output in the second cycle through the second visual stimulus image may comprise controlling the synthesis coefficient multiplied to the first visual stimulus image and an output frequency of the motion image, comprising determining a first frequency of the first visual stimulus through the synthesis coefficient, by the processor and determining the output frequency of the motion image as a second frequency that is the same as or different from the first frequency, by the processor.

According to an exemplary embodiment, the simultaneously presenting the first visual stimulus that blinks the target region at the first cycle and the second visual stimulus in which the motion image is sequentially output in the second cycle through the second visual stimulus image may comprise variably determining a first frequency for determining the first cycle and a second frequency for determining the second cycle, depending on a presentation purpose of the second visual stimulus image.

According to an exemplary embodiment, the variably determining the first frequency for determining the first cycle and the second frequency for determining the second cycle, depending on the presentation purpose of the second visual stimulus image may comprise determining the first frequency to be greater than the second frequency, when a presentation purpose of the first visual stimulus is purposed to induce steady-state visual evoked potential (SSVEP) and the presentation purpose of the second visual stimulus is purposed to induce concentration, and the simultaneously presenting the first visual stimulus that blinks the target region at the first cycle and the second visual stimulus in which the motion image is sequentially output in the second cycle through the second visual stimulus image comprises providing a notification to induce concentration, when an SSVEP or steady-state motion visual evoked potential (SSMVEP) response size according to the second visual stimulus is smaller than or equal to a preset criterion, by the processor.

According to an aspect of the present disclosure, a brain-computer interface (BCI)-based visual stimulus presentation device for active observation rehabilitation is provided. The device may comprise a computing device comprising a processor and a memory. The memory may be configured to store instructions that, when executed by the processor, are configured to cause the processor to obtain a motion image from a video, generate a first visual stimulus image divided into a target region and a background region, generate a second visual stimulus image by synthesizing the motion image and the first visual stimulus image, and simultaneously present the first visual stimulus that blinks the target region at a first cycle and the second visual stimulus in which the motion image is sequentially output in a second cycle through the second visual stimulus image.

According to an exemplary embodiment, the obtaining the motion image from the video may comprise extracting a plurality of motion images over time from a motion video photographed for action observation (AO).

According to an exemplary embodiment, the first visual stimulus image may be generated through image segmentation based on the motion image, and the second visual stimulus image may be generated through image blending.

According to an exemplary embodiment, the generating the first visual stimulus image divided into the target region and the background region through image segmentation based on the motion image may comprise storing position information of the target region and changing a color of the target region to a different color from a color of the background region.

According to an exemplary embodiment, the generating the second visual stimulus image by synthesizing the motion image and the first visual stimulus image through image blending may comprise writing shader code for the image blending, using X_new=X_org+αX_blend. X_new is the second visual stimulus image, X_org is the motion image, X_blend is the first visual stimulus image, and α is a synthesis coefficient.

According to an exemplary embodiment, the generating the second visual stimulus image by synthesizing the motion image and the first visual stimulus image through image blending further may comprise enabling the second visual stimulus image to output the motion image as is when α is 0, and enabling the second visual stimulus image to brightly display only the target region when α is 1.

According to an exemplary embodiment, the simultaneously presenting the first visual stimulus that blinks the target region at the first cycle and the second visual stimulus in which the motion image is sequentially output in the second cycle through the second visual stimulus image may comprise controlling the synthesis coefficient multiplied to the first visual stimulus image and an output frequency of the motion image.

According to an exemplary embodiment, the controlling the synthesis coefficient multiplied to the first visual stimulus image and the output frequency of the motion image may comprise determining a first frequency of the first visual stimulus through the synthesis coefficient and determining the output frequency of the motion image as a second frequency that is the same as or different from the first frequency.

According to an exemplary embodiment, the simultaneously presenting the first visual stimulus that blinks the target region at the first cycle and the second visual stimulus in which the motion image is sequentially output in the second cycle through the second visual stimulus image may comprise variably determining a first frequency for determining the first cycle and a second frequency for determining the second cycle, depending on a presentation purpose of the second visual stimulus image.

According to an exemplary embodiment, the variably determining the first frequency for determining the first cycle and the second frequency for determining the second cycle, depending on the presentation purpose of the second visual stimulus image may comprise determining the first frequency to be greater than the second frequency, when a presentation purpose of the first visual stimulus is purposed to induce steady-state visual evoked potential (SSVEP) and the presentation purpose of the second visual stimulus is purposed to induce concentration.

According to an exemplary embodiment, the simultaneously presenting the first visual stimulus that blinks the target region at the first cycle and the second visual stimulus in which the motion image is sequentially output in the second cycle through the second visual stimulus image further may comprise providing a notification to induce concentration, when an SSVEP or steady-state motion visual evoked potential (SSMVEP) response size according to the second visual stimulus is smaller than or equal to a preset criterion.

### BRIEF DESCRIPTION OF THE DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1 illustrates a block diagram of a BCI-based visual stimulus presentation device, according to an exemplary embodiment of the present disclosure.

FIG. 2 illustrates a flowchart of a BCI-based visual stimulus presentation method, according to an exemplary embodiment of the present disclosure.

FIG. 3 illustrates a block diagram of a BCI-based visual stimulus presentation method, according to an exemplary embodiment of the present disclosure.

FIG. 4 illustrates a drawing for explaining a visual stimulus preparation step, according to an exemplary embodiment of the present disclosure.

FIG. 5 illustrates a drawing for explaining a visual stimulus synthesis step, according to an exemplary embodiment of the present disclosure.

FIG. 6 illustrates a graph for explaining a visual stimulus presentation step, according to an exemplary embodiment of the present disclosure.

FIG. 7 illustrates a drawing for explaining a visual stimulus presentation step, according to an exemplary embodiment of the present disclosure.

FIG. 8 to FIG. 10 illustrate graphs for explaining the effect of embodiments of the present disclosure.

FIG. 11 illustrates a drawing for explaining a computing device, according to an exemplary embodiment of the present disclosure.

### DETAILED DESCRIPTION

An embodiment of the disclosure will be described more fully hereinafter with reference to the accompanying drawings such that a person skill in the art may easily implement the embodiment. As those skilled in the art would realize, the described embodiments may be modified in various different ways, all without departing from the spirit or scope of the present disclosure. In order to clarify the present disclosure, parts that are not related to the description will be omitted, and the same elements or equivalents are referred to with the same reference numerals throughout the specification.

In addition, unless explicitly described to the contrary, the word “comprise” and variations such as “comprises” or “comprising” will be understood to imply the inclusion of stated elements but not the exclusion of any other elements. Terms including an ordinary number, such as first and second, are used for describing various constituent elements, but the constituent elements are not limited by the terms. The terms are only used to differentiate one component from other components.

In addition, the terms “unit”, “part” or “portion”, “-er”, and “module” in the specification refer to a unit that processes at least one function or operation, which may be implemented by hardware, software, or a combination of hardware and software.

Additionally, at least some of the configurations or functions of a BCI-based visual stimulus presentation device and method according to the embodiments described below may be implemented as a program or software, and the program or software may be stored in a computer-readable medium.

In describing components of embodiments of the present disclosure, the terms first, second, A, B, (a), (b), and the like may be used herein. These terms are only used to distinguish one component from another component, but do not limit the corresponding components irrespective of the nature, order, or priority of the corresponding components. Furthermore, unless otherwise defined, all terms including technical and scientific terms used herein are to be interpreted as is customary in the art to which the present disclosure belongs. Such terms as those defined in a generally used dictionary are to be interpreted as having meanings equal to the contextual meanings in the relevant field of art, and are not to be interpreted as having ideal or excessively formal meanings unless clearly defined as having such in the present application.

In addition, terms such as first, second, and the like used herein may be used to describe various components, but the various components are not limited by these terms. The terms are used solely for the purpose of distinguishing one component from other components. For example, a first component may be referred to as a second component, and a second component may also be referred to as a first component, without departing from the scope of rights according to the inventive concepts of the present disclosure.

The following Detailed Description is merely provided by way of example and not of limitation. Furthermore, there is no intention to be bound by any expressed or implied theory presented in the preceding background or in the following Detailed Description.

Reference will now be made in detail to various exemplary embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications, and equivalents, which may be included within the spirit and scope of the various embodiments as defined by the appended claims. Furthermore, in this Detailed Description, numerous specific details are set forth in order to provide a thorough understanding of embodiments of the present subject matter. However, embodiments may be practiced without these specific details. In other instances, well-known methods, procedures, components, and circuits have not been described in detail as not to unnecessarily obscure aspects of the described embodiments.

Some portions of the detailed descriptions which follow are presented in terms of procedures, logic blocks, processing, and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities may take the form of electrical or magnetic signals capable of being stored, transferred, combined, compared, and otherwise manipulated in an electronic system, device, and/or component.

It should be borne in mind, however, that these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “determining,” “communicating,” “taking,” “comparing,” “monitoring,” “calibrating,” “estimating,” “initiating,” “providing,” “receiving,” “controlling,” “transmitting,” “isolating,” “generating,” “aligning,” “synchronizing,” “identifying,” “maintaining,” “displaying,” “switching,” or the like, refer to the actions and processes of an electronic item such as: a processor, a sensor processing unit (SPU), a processor of a sensor processing unit, an application processor of an electronic device/system, or the like, or a combination thereof. The item manipulates and transforms data represented as physical (electronic and/or magnetic) quantities within the registers and memories into other data similarly represented as physical quantities within memories or registers or other such information storage, transmission, processing, or display components.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example device vibration sensing system and/or electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration. One or more components of an SPU or electronic device described herein may be embodied in the form of one or more of a “chip,” a “package,” an Integrated Circuit (IC).

Hereinafter, various embodiments disclosed in the present disclosure will be described in detail with reference to the accompanying drawings, and the same or similar elements are designated by the same reference numerals regardless of the numerals in the drawings and redundant description thereof will be omitted.

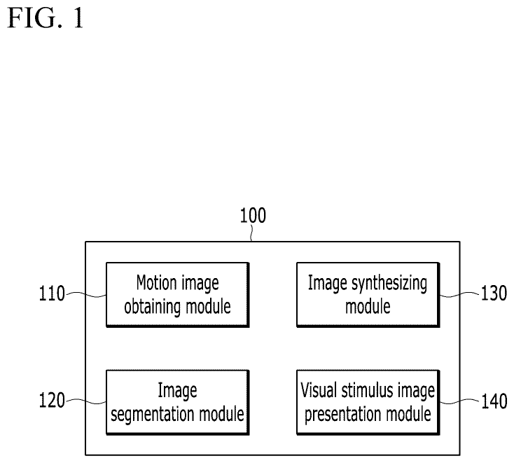

FIG. 1 illustrates a block diagram of a BCI-based visual stimulus presentation device, according to an exemplary embodiment of the present disclosure.

Referring to FIG. 1, a BCI-based visual stimulus presentation device 100, according to an exemplary embodiment, may be configured to execute program codes or instructions loaded in one or more memory devices through one or more processors.

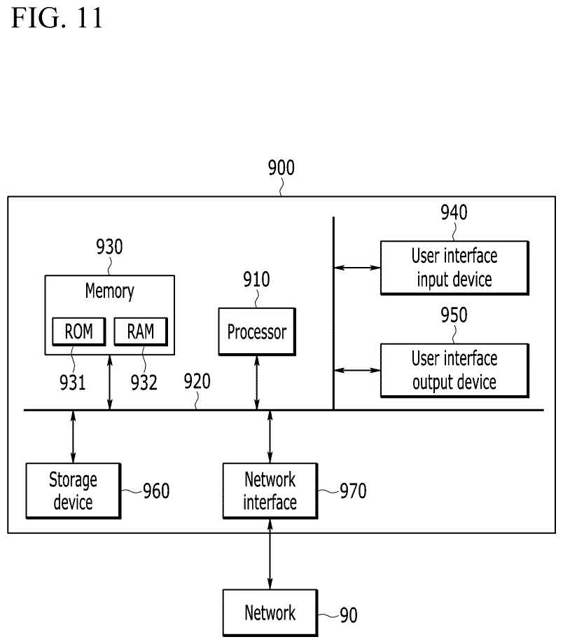

For example, the BCI-based visual stimulus presentation device 100 may be implemented as a computing device 900 described later with reference to FIG. 11. In this case, the one or more processors may correspond to a processor 910 of the computing device 900, and the one or more memory devices may correspond to a memory 930 of the computing device 900.

The program codes or instructions may be executed by the one or more processors, to induce steady-state visual evoked potential (SSVEP)/steady-state motion visual evoked potential (SSMVEP) only when the patient looks at the motion part required for rehabilitation, and may be configured to identify the rehabilitation concentration of the patient by utilizing SSVEP/SSMVEP responsiveness. In this disclosure, the term “module” is used to logically differentiate functions performed by the program code or instruction.

Referring to FIG. 1, the BCI-based visual stimulus presentation device 100 may comprise a motion image obtaining module 110, an image segmentation module 120, an image synthesizing module 130 and a visual stimulus image presentation module 140.

The motion image obtaining module 110 may be configured to obtain a motion image from a video.

The motion image obtaining module 110 may be configured to extract a plurality of motion images over time from a motion video photographed for action observation (AO).

The image segmentation module 120 may be configured to generate a first visual stimulus image divided into a target region and a background region through image segmentation based on the motion image.

The image segmentation module 120 may be configured to store position information of the target region.

The image segmentation module 120 may be configured to change a color of the target region to a different color from a color of the background region.

The image synthesizing module 130 may be configured to generate a second visual stimulus image by synthesizing the motion image and the first visual stimulus image through image blending.

The image synthesizing module 130 may be configured to write shader code including, e.g., Equation 1 below, for image blending.

$${X}_{new} = {X}_{org} + α {X}_{blend} \qquad Equation 1$$

Here, X_{new} is the second visual stimulus image, X_{org} is the motion image, X_{blend} is the first visual stimulus image, and α is a synthesis coefficient.

The image synthesizing module 130 may be configured to enable the second visual stimulus image to output the motion image as is when α is 0, and may be configured to enable the second visual stimulus image to brightly display only the target region when α is 1.

The visual stimulus image presentation module 140 may be configured to simultaneously present the first visual stimulus that blinks the target region at a first cycle and the second visual stimulus in which the motion image is sequentially output in a second cycle through the second visual stimulus image.

The visual stimulus image presentation module 140 may be configured to control the synthesis coefficient multiplied to the first visual stimulus image and an output frequency of the motion image.

The visual stimulus image presentation module 140 may be configured to determine a first frequency of the first visual stimulus through the synthesis coefficient.

The visual stimulus image presentation module 140 may be configured to determine the output frequency of the motion image as a second frequency that is the same as or different from the first frequency.

The visual stimulus image presentation module 140 may be configured to variably determine the first frequency for determining the first cycle and the second frequency for determining the second cycle, depending on a presentation purpose of the second visual stimulus image.

When a presentation purpose of the first visual stimulus is purposed to induce SSVEP and the presentation purpose of the second visual stimulus is purposed to induce concentration, the visual stimulus image presentation module 140 may be configured to determine the first frequency to be greater than the second frequency.

When an SSVEP response size according to the second visual stimulus is smaller than or equal to a preset criterion, the visual stimulus image presentation module 140 may be configured to provide a notification to induce concentration.

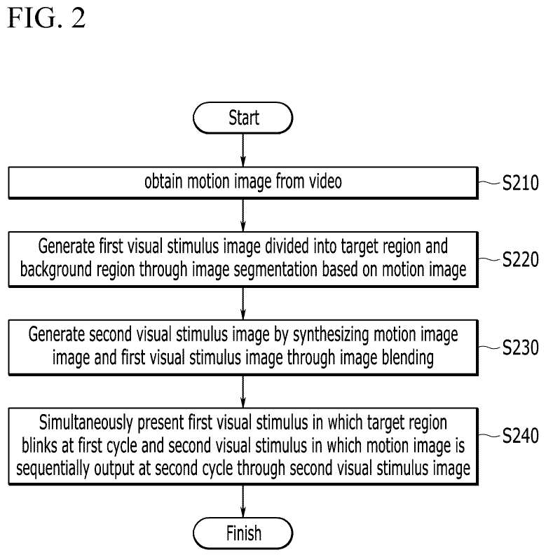

FIG. 2 illustrates a flowchart of a BCI-based visual stimulus presentation method, according to an exemplary embodiment of the present disclosure. The BCI-based visual stimulus presentation method of FIG. 2 may be configured to be performed through the BCI-based visual stimulus presentation device 100 of FIG. 1.

In FIG. 2, the BCI-based visual stimulus presentation device 100 may be configured to obtain the motion image from a video, at step S210.

The BCI-based visual stimulus presentation device 100 may be configured to generate the first visual stimulus image divided into the target region and the background region through image segmentation based on the motion image, at step S220.

The BCI-based visual stimulus presentation device 100 may be configured to synthesize the motion image and the first visual stimulus image through image blending, to generate the second visual stimulus image, at step S230.

The BCI-based visual stimulus presentation device 100 may be configured to simultaneously present the first visual stimulus that blinks the target region at the first cycle and the second visual stimulus in which the motion image is sequentially output in the second cycle through the second visual stimulus image, at step S240.

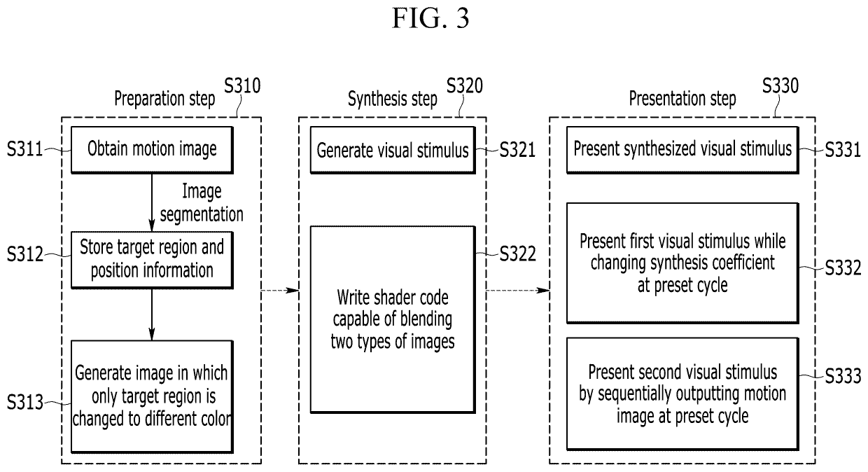

FIG. 3 illustrates a block diagram of the BCI-based visual stimulus presentation method, according to an exemplary embodiment of the present disclosure. The BCI-based visual stimulus presentation method of FIG. 3 may be configured to be performed through the BCI-based visual stimulus presentation device 100 of FIG. 1.

In FIG. 3, the BCI-based visual stimulus presentation method may comprise a preparation step (step S310), a synthesizing step (step S320), and a presentation step (step S330).

In the preparation step (the step S310), the BCI-based visual stimulus presentation device 100 may be configured to extract the plurality of motion images over time from the motion video photographed for action observation (AO), at step S311.

The BCI-based visual stimulus presentation device 100 may be configured to change the color of the target region to a different color from the color of the background region through image segmentation using a SAM model, or the like, and may be configured to store position information of the target region, at step S312.

The BCI-based visual stimulus presentation device 100 may be configured to generate the first visual stimulus image in which only the target region is changed to a different color from other regions, at step S313.

In the synthesis step (the step S320), the BCI-based visual stimulus presentation device 100 may be configured to synthesize the first visual stimulus image and the motion image, to generate the second visual stimulus image, at step S321.

The BCI-based visual stimulus presentation device 100 may be configured to write shader code through Equation 1 below, for image blending, at step S322.

The shader code may be a function in computer graphics that calculates the position and color of a pixel to be ultimately output to the screen, according to Equation 1.

Here, X_{new} is the synthesized second visual stimulus image, X_{org} is the motion image, X_{blend} is the first visual stimulus image, and α is the synthesis coefficient.

That is, the BCI-based visual stimulus presentation device 100 may be configured to output the motion image as is when the synthesis coefficient is 0, and may be configured to output it to brighten target portion when the synthesis coefficient is 1.

In the presentation step (the step S330), the BCI-based visual stimulus presentation device 100 may be configured to present the synthesized second visual stimulus image, at step S331.

The BCI-based visual stimulus presentation device 100 may be configured to determine the first frequency of the first visual stimulus through the synthesis coefficient.

The BCI-based visual stimulus presentation device 100 may be configured to determine the output frequency of the motion image as the second frequency that is the same as or different from the first frequency of the first visual stimulus.

The BCI-based visual stimulus presentation device 100 may be configured to change the synthesis coefficient or a blending control parameter of the image blending at a regular cycle, at step S332.

For example, the BCI-based visual stimulus presentation device 100 may be configured to induce SSVEP including a 6 Hz component through visual stimulus that illuminates only the hand portion every ⅙ second, by controlling the parameter in a square waveform with a cycle of ⅙ second.

The BCI-based visual stimulus presentation device 100 may be configured to control the output of the motion image, at step S333.

The BCI-based visual stimulus presentation device 100 may be configured to sequentially present the motion image at a preset cycle.

For example, the BCI-based visual stimulus presentation device 100 may be configured to sequentially present 18 images of hand operations every ⅙ second, to induce the SSMVEP including the 6 Hz component.

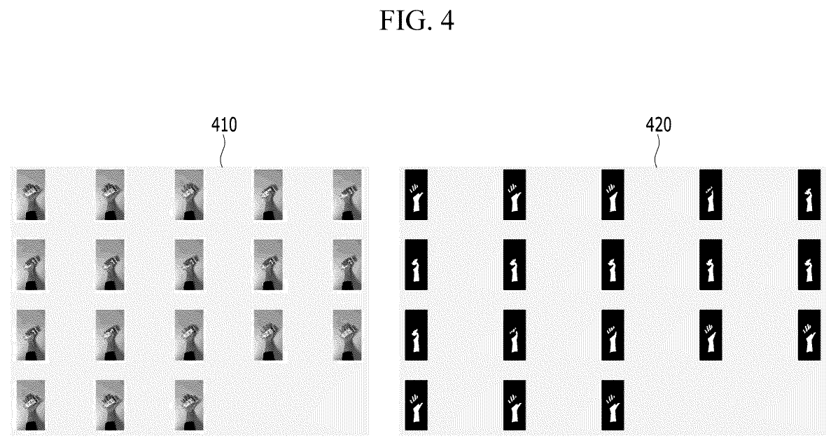

FIG. 4 illustrates a drawing for explaining the visual stimulus preparation step, according to an exemplary embodiment of the present disclosure.

In FIG. 4, the BCI-based visual stimulus presentation device 100 may be configured to obtain the motion image to be used for action observation rehabilitation.

For example, the BCI-based visual stimulus presentation device 100 may be configured to extract 18 motion images 410 after photographing a hand grip operation.

The BCI-based visual stimulus presentation device 100 may be configured to perform image segmentation on each image, and separate it into two groups of a rehabilitation target portion (e.g., the hand and the arm) and a portion other than that.

If the segmentation performance is poor, groups may be separated manually later.

The BCI-based visual stimulus presentation device 100 may be configured to generate an image 420 composed of two colors by adding a specific color to the pixel position of rehabilitation target portion and adding the black color to the other portion.

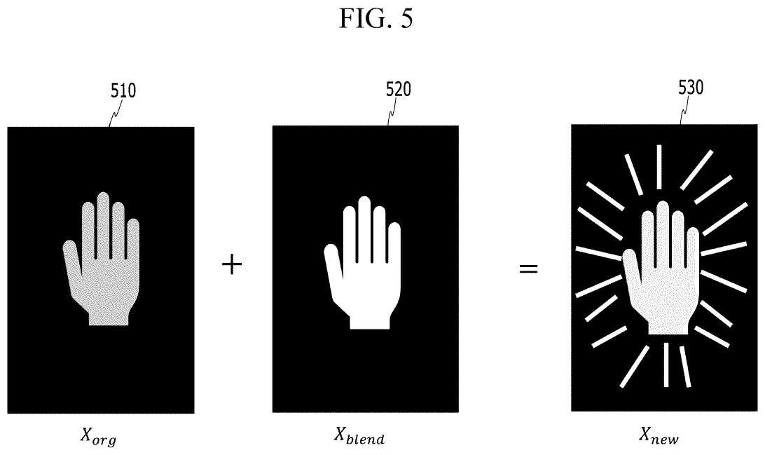

FIG. 5 illustrates a drawing for explaining a visual stimulus synthesis step, according to an exemplary embodiment of the present disclosure.

The BCI-based visual stimulus presentation device 100 may be configured to use float3(R, G, B) for color value calculation and/or other suitable means.

The BCI-based visual stimulus presentation device 100 may be configured to generate the visual stimulus image synthesized by using Equation 1.

Here, X_{new} is the synthesized second visual stimulus image 530, X_{org} is the original motion image 510, X_{blend} is an image 520 in which only the target portion is in white color (1, 1, 1) and others are in black color (0, 0, 0) through segmentation, and α is a brightness synthesis coefficient float value (0 to 1).

The BCI-based visual stimulus presentation device 100 may be configured to output the original image as is (X_{new}=X_{org}) when α is 0.

When α is 1, the BCI-based visual stimulus presentation device 100 may be configured to brightly display only the target portion by adding (1, 1, 1) to only the target portion.

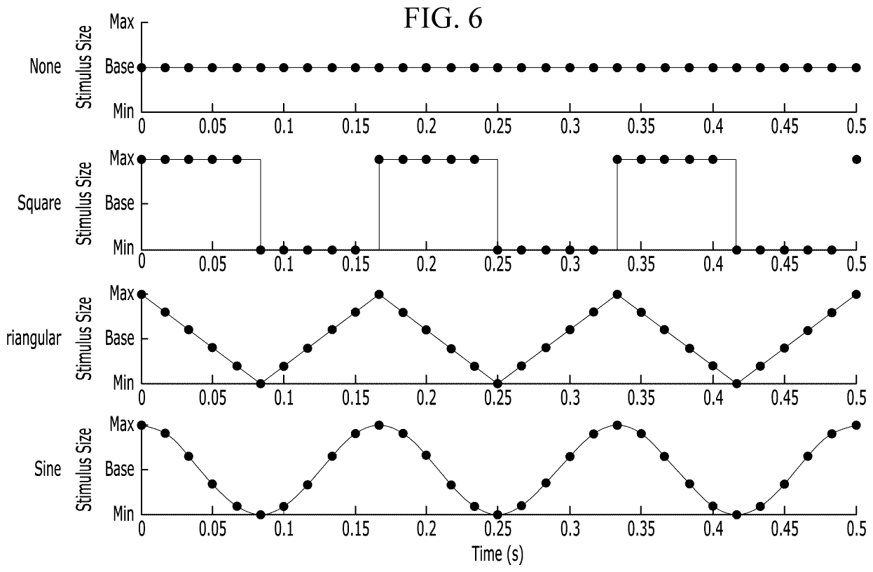

FIG. 6 illustrates a graph for explaining the visual stimulus presentation step, according to an exemplary embodiment of the present disclosure.

In FIG. 6, the BCI-based visual stimulus presentation device 100 may be configured to adjust the value of a blending parameter in various ways.

For example, the BCI-based visual stimulus presentation device 100 may be configured to change the brightness synthesis coefficient α in a square waveform with a cycle of 1/4.5 second.

The BCI-based visual stimulus presentation device 100 may be configured to generate an image of a hand glowing at a rate of 4.5 Hz.

The BCI-based visual stimulus presentation device 100 may be configured to evoke SSVEP of 4.5 Hz component through stimulation blinking at 4.5 Hz.

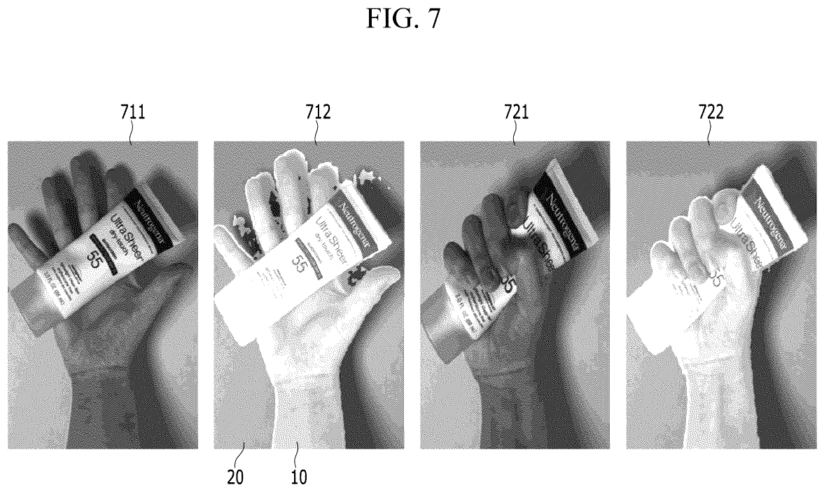

FIG. 7 illustrates a drawing for explaining the visual stimulus presentation step, according to an exemplary embodiment of the present disclosure.

The BCI-based visual stimulus presentation device 100 may be configured to control the blending parameter and the motion image output in the stimulus presentation step.

The BCI-based visual stimulus presentation device 100 may be configured to generate visual stimulus images 712 and 722 that illuminate only the target region 10 except for the background region 20 by controlling the brightness synthesis coefficient in the original motion images 711 and 721.

The BCI-based visual stimulus presentation device 100 may be configured to sequentially output a first motion image 711 and a second motion image 721 at a regular interval.

For example, the BCI-based visual stimulus presentation device 100 may be configured to sequentially output the 18 motion images including the first and second motion images 711 and 721 every ⅕ second.

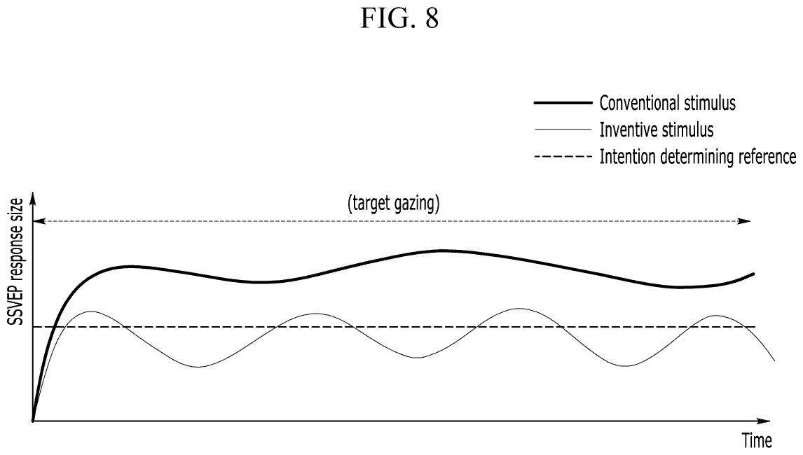

FIG. 8 to FIG. 10 illustrate graphs for explaining the effect of embodiments of the present disclosure.

The BCI-based visual stimulus presentation device 100 may be configured to evoke SSVEP/SSMVEP only when the patient is looking at the motion part necessary for rehabilitation.

The BCI-based visual stimulus presentation device 100 may be configured to determine whether the patient perform the action observation rehabilitation well.

The BCI-based visual stimulus presentation device 100 may be configured to evoke a weak response SSVEP/SSMVEP, when the patient does not concentrate on the motion part or gazes the background.

The BCI-based visual stimulus presentation device 100 may be configured to indirectly determine the patient's rehabilitation concentration by utilizing SSVEP/SSMVEP responsiveness.

The BCI-based visual stimulus presentation device 100 may be configured to overcome the weak response of SSMVEP through SSVEP stimulation that blinks only the target portion.

FIG. 8 illustrates a graph showing the difference in effect between the conventional visual stimulus (the Comparative Example) in which the background region blinks and a visual stimulus, according to an exemplary embodiment of the present disclosure, in the original image including the background region and the target region (e.g., the hand), with the SSVEP response size as an indicator.

In FIG. 8, in the case of the conventional visual stimulus, even when the patient gazes the background, it may be classified as having a rehabilitation intention since the SSVEP response is evoked by more than an intention determining reference.

In the present embodiment, in the case where the patient gazes the background, the SSVEP has a response size smaller than or equal to the intention determining reference so it may be classified as not having the rehabilitation intention.

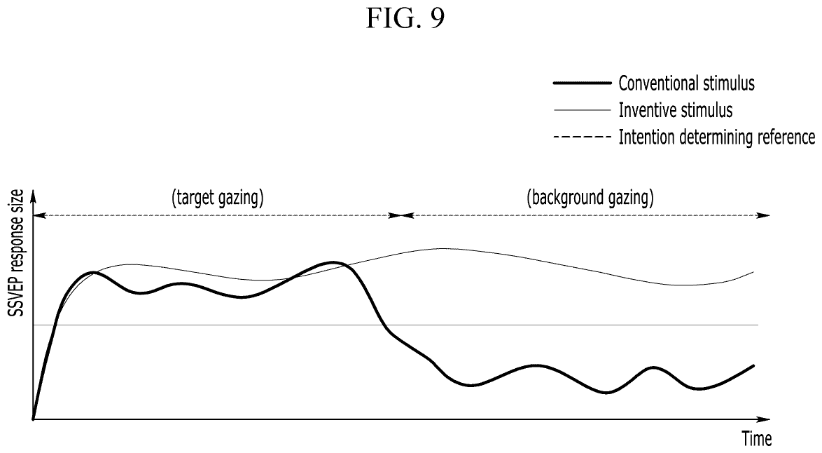

FIG. 9 illustrates a graph showing the difference in effect between the conventional visual stimulus (the Comparative Example) that evokes SSMVEP only by the hand motion image and the visual stimulus of the present disclosure, according to an exemplary embodiment of the present disclosure.

In FIG. 9, in the case of the conventional stimulus, the size of SSMVEP response sometimes falls short of the intention determining reference, so that the rehabilitation intention is sometimes not properly classified.

In the present disclosure, when the patient gazes the hand operation, the size of segmented SSVEP response is sufficient and the rehabilitation intention is accurately classified.

That is, in the case of the Comparative Example, the response is not large because it is evoked only by SSMVEP, but in the case of the embodiment, SSMVEP/SSVEP (frequency matching) are evoked together so that the size of response is sufficiently large.

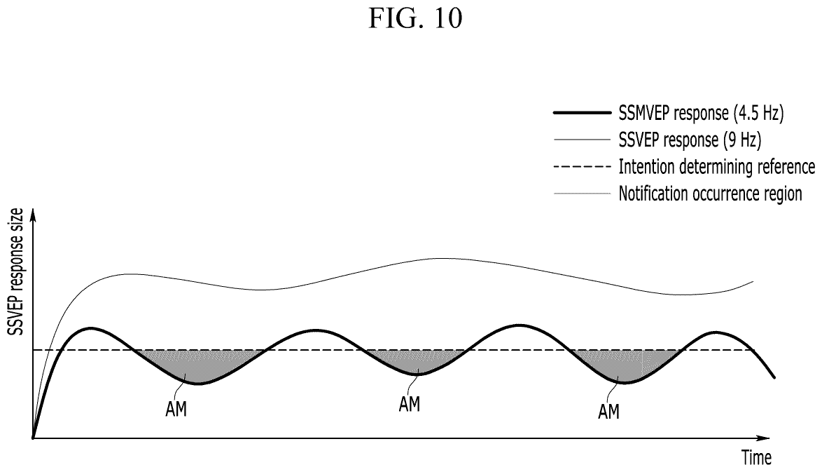

FIG. 10 illustrates a graph showing the effect when the first frequency of the first visual stimulus evoking SSVEP and the second frequency of the second visual stimulus evoking SSMVEP in a synthetic visual stimulus image are set to be different, according to an exemplary embodiment of the present disclosure.

In FIG. 10, the BCI-based visual stimulus presentation device 100 may be configured to variably determine the first frequency for determining the first cycle of the first visual stimulus and the second frequency for determining the second cycle of the second visual stimulus, according to the presentation purpose of the synthetic visual stimulus image.

For example, when the presentation purpose of the first visual stimulus is purposed to induce SSVEP and the presentation purpose of the second visual stimulus is purposed to induce concentration, the BCI-based visual stimulus presentation device 100 may be configured to determine the first frequency to be greater than the second frequency.

When an SSMVEP response size according to the second visual stimulus is smaller than or equal to a preset criterion, the BCI-based visual stimulus presentation device 100 may be configured to provide the notification to induce concentration.

For example, when the size of SSMVEP response according to the second visual stimulus falls short of the intention determining reference, the BCI-based visual stimulus presentation device 100 may be configured to designate it as a notification occurrence region (AM), and may be configured to provide a notification to the patient.

FIG. 10 shows the result when the patient gazes the hand operation through a graph, when the frequency of the first visual stimulus is 9 Hz and the frequency of the second visual stimulus is 4.5 Hz.

That is, in an embodiment, SSVEP of the first visual stimulus having relatively large reactivity may be used for rehabilitation purpose, and SSMVEP of the second visual stimulus having relatively small reactivity may be used for the notification to induce concentration.

FIG. 11 illustrates a drawing for explaining a computing device, according to an exemplary embodiment of the present disclosure.

Referring to FIG. 11, a BCI-based visual stimulus presentation device and method according to embodiments may be implemented by using the computing device 900.

The computing device 900 may comprise at least one of a processor 910, a memory 930, the user interface input device 940, the user interface output device 950 and a storage device 960 that communicate through a bus 920. The computing device 900 may also comprise a network interface 970 electrically connected to a network 90. The network interface 970 may be configured to transmit or receive signals with other entities through the network 90.

The processor 910 may be implemented in various types such as a micro controller unit (MCU), an application processor (AP), a central processing unit (CPU), a graphic processing unit (GPU), a neural processing unit (NPU), and the like, and may be any type of semiconductor device capable of executing instructions stored in the memory 930 or the storage device 960. The processor 910 may be configured to implement the functions and methods described above with respect to FIG. 1 to FIG. 10.

The memory 930 and the storage device 960 may comprise various types of volatile or non-volatile storage media. For example, the memory may comprise a read-only memory (ROM) 931 and random-access memory (RAM) 932. In this embodiment, the memory 930 may be located inside or outside processor 910, and the memory 930 may be connected to the processor 910 through various known means.

In some embodiments, at least some configurations or functions of the BCI-based visual stimulus presentation device and method according to an embodiment may be implemented as a program or software executable by the computing device 900, and program or software may be stored in a computer-readable medium.

In some embodiments, at least some configurations or functions of the BCI-based visual stimulus presentation device and method according to an embodiment may be implemented by using hardware or circuitry of the computing device 900, or may also be implemented as separate hardware or circuitry that may be electrically connected to the computing device 900.

While this disclosure has been described in connection with what is presently considered to be practical embodiments, it is to be understood that the disclosure is not limited to the disclosed embodiments, but, on the contrary, is intended to cover various modifications and equivalent arrangements included within the spirit and scope of the appended claims.

What is claimed is:

1. A brain-computer interface (BCI)-based visual stimulus presentation method, comprising:
using a computing device comprising a processor and a memory:
obtaining a motion image from a video;
generating a first visual stimulus image divided into a target region and a background region;
generating a second visual stimulus image by synthesizing the motion image and the first visual stimulus image; and
simultaneously presenting the first visual stimulus that blinks the target region at a first cycle and the second visual stimulus in which the motion image is sequentially output in a second cycle through the second visual stimulus image.

2. The BCI-based visual stimulus presentation method of claim 1, wherein the obtaining the motion image from the video comprises extracting a plurality of motion images over time from a motion video photographed for action observation (AO).

3. The BCI-based visual stimulus presentation method of claim 1, wherein:
the first visual stimulus image is generated through image segmentation based on the motion image, and
the second visual stimulus image is generated through image blending.

4. The BCI-based visual stimulus presentation method of claim 3, wherein the generating the first visual stimulus image divided into the target region and the background region through image segmentation based on the motion image comprises:
storing position information of the target region; and
changing a color of the target region to a different color from a color of the background region.

5. The BCI-based visual stimulus presentation method of claim 3, wherein the generating the second visual stimulus image by synthesizing the motion image and the first visual stimulus image through image blending comprises:
writing shader code for the image blending, by the processor, using X_{new}=X_{org}+αX_{blend},
wherein, X_{new} is the second visual stimulus image, X_{org} is the motion image, X_{blend} is the first visual stimulus image, and α is a synthesis coefficient.

6. The BCI-based visual stimulus presentation method of claim 5, wherein the generating the second visual stimulus image by synthesizing the motion image and the first visual stimulus image through image blending further comprises:
enabling the second visual stimulus image to output the motion image as is when α is 0; and
enabling the second visual stimulus image to brightly display only the target region when α is 1.

7. The BCI-based visual stimulus presentation method of claim 6, wherein the simultaneously presenting the first visual stimulus that blinks the target region at the first cycle and the second visual stimulus in which the motion image is sequentially output in the second cycle through the second visual stimulus image comprises:
controlling the synthesis coefficient multiplied to the first visual stimulus image and an output frequency of the motion image, comprising:
determining a first frequency of the first visual stimulus through the synthesis coefficient, by the processor; and
determining the output frequency of the motion image as a second frequency that is the same as or different from the first frequency, by the processor.

8. The BCI-based visual stimulus presentation method of claim 1, wherein the simultaneously presenting the first visual stimulus that blinks the target region at the first cycle and the second visual stimulus in which the motion image is sequentially output in the second cycle through the second visual stimulus image comprises:
variably determining a first frequency for determining the first cycle and a second frequency for determining the second cycle, depending on a presentation purpose of the second visual stimulus image.

9. The BCI-based visual stimulus presentation method of claim 8, wherein:
the variably determining the first frequency for determining the first cycle and the second frequency for determining the second cycle, depending on the presentation purpose of the second visual stimulus image comprises:
determining the first frequency to be greater than the second frequency, when a presentation purpose of the first visual stimulus is purposed to induce steady-state visual evoked potential (SSVEP) and the presentation purpose of the second visual stimulus is purposed to induce concentration, and
the simultaneously presenting the first visual stimulus that blinks the target region at the first cycle and the second visual stimulus in which the motion image is sequentially output in the second cycle through the second visual stimulus image comprises:
providing a notification to induce concentration, when an SSVEP or steady-state motion visual evoked potential (SSMVEP) response size according to the second visual stimulus is smaller than or equal to a preset criterion, by the processor.

10. A brain-computer interface (BCI)-based visual stimulus presentation device for active observation rehabilitation, comprising:
a computing device comprising a processor and a memory, wherein the memory is configured to store instructions that, when executed by the processor, are configured to cause the processor to:
obtain a motion image from a video;
generate a first visual stimulus image divided into a target region and a background region;
generate a second visual stimulus image by synthesizing the motion image and the first visual stimulus image; and
simultaneously present the first visual stimulus that blinks the target region at a first cycle and the second visual stimulus in which the motion image is sequentially output in a second cycle through the second visual stimulus image.

11. The BCI-based visual stimulus presentation device of claim 10, wherein the obtaining the motion image from the video comprises:
extracting a plurality of motion images over time from a motion video photographed for action observation (AO).

12. The BCI-based visual stimulus presentation device of claim 10, wherein:
the first visual stimulus image is generated through image segmentation based on the motion image, and
the second visual stimulus image is generated through image blending.

13. The BCI-based visual stimulus presentation device of claim 12, wherein the generating the first visual stimulus image divided into the target region and the background region through image segmentation based on the motion image comprises:
storing position information of the target region; and
changing a color of the target region to a different color from a color of the background region.

14. The BCI-based visual stimulus presentation device of claim 12, wherein the generating the second visual stimulus image by synthesizing the motion image and the first visual stimulus image through image blending comprises:
writing shader code for the image blending, using X_{new}=X_{org}+αX_{blend},
wherein X_{new} is the second visual stimulus image, X_{org} is the motion image, X_{blend} is the first visual stimulus image, and α is a synthesis coefficient.

15. The BCI-based visual stimulus presentation device of claim 14, wherein the generating the second visual stimulus image by synthesizing the motion image and the first visual stimulus image through image blending further comprises:
enabling the second visual stimulus image to output the motion image as is when α is 0; and
enabling the second visual stimulus image to brightly display only the target region when α is 1.

16. The BCI-based visual stimulus presentation device of claim 15, wherein the simultaneously presenting the first visual stimulus that blinks the target region at the first cycle and the second visual stimulus in which the motion image is sequentially output in the second cycle through the second visual stimulus image comprises:
controlling the synthesis coefficient multiplied to the first visual stimulus image and an output frequency of the motion image.

17. The BCI-based visual stimulus presentation device of claim 16, wherein the controlling the synthesis coefficient multiplied to the first visual stimulus image and the output frequency of the motion image comprises:
determining a first frequency of the first visual stimulus through the synthesis coefficient; and
determining the output frequency of the motion image as a second frequency that is the same as or different from the first frequency.

18. The BCI-based visual stimulus presentation device of claim 11, wherein the simultaneously presenting the first visual stimulus that blinks the target region at the first cycle and the second visual stimulus in which the motion image is sequentially output in the second cycle through the second visual stimulus image comprises:
variably determining a first frequency for determining the first cycle and a second frequency for determining the second cycle, depending on a presentation purpose of the second visual stimulus image.

19. The BCI-based visual stimulus presentation device of claim 18, wherein the variably determining the first frequency for determining the first cycle and the second frequency for determining the second cycle, depending on the presentation purpose of the second visual stimulus image comprises:
determining the first frequency to be greater than the second frequency, when a presentation purpose of the first visual stimulus is purposed to induce steady-state visual evoked potential (SSVEP) and the presentation purpose of the second visual stimulus is purposed to induce concentration.

20. The BCI-based visual stimulus presentation device of claim 19, wherein the simultaneously presenting the first visual stimulus that blinks the target region at the first cycle and the second visual stimulus in which the motion image is sequentially output in the second cycle through the second visual stimulus image further comprises:
providing a notification to induce concentration, when an SSVEP or steady-state motion visual evoked potential (SSMVEP) response size according to the second visual stimulus is smaller than or equal to a preset criterion.
